(19) United States
(12) Patent Application Publication
(10) Pub. No.: US 2005/0033317 A1
(43) Pub. Date: Feb. 10, 2005

(54) "Staple align", medical skin stapler with attached skin tissue forceps

(76) Inventor: Claudette M. Ables, Albia, IA (US)

Correspondence Address: CLAUDETTE M. ABLES, 20 1/2 NORTH MAIN STREET, APT #4, ALBIA, IA, 52531, US

(21) Appl. No.: 10/761,647

(22) Filed: Jan. 20, 2004

Related U.S. Application Data
(60) Provisional application No. 60/486,551, filed on Jul. 11, 2003.

(51) Int. Cl. A61B 17/10

(52) U.S. Cl. 606/139

(57) ABSTRACT

The prior art discloses a medical skin stapler having a means for housing, forming, activating, and impregnating sterile staple into skin with the grasping and squeezing of the hand held stapler with manual power to the trigger; U.S. Pat. Nos. 4,391,402, 4,406,392, 4,458,835, 4,591,086, and DES 269,459. A new design improvement for medical skin stapler has been invented. The control means are contained in surgical instrument. The control means comprise of a double toothed and single toothed forcep on each side nose bottom of skin stapler. On partially compressing the trigger prior to stapling. The forceps will grasp skin bilaterally simultaneously with equal tension to disunited skin for precise proximation to skin. On completely compressing the trigger the stapling is completed. A release of trigger will achieve stapling and release of skin.

### CROSS REFERENCE TO RELATED APPLICATIONS

This application is based upon: Provisional Application #60/486,551

### BACKGROUND OF THE INVENTION

It is well known in the medical field that skin staplers are used for closure of skin from incisional and or injuries to disunited skin. In the prior art, there are no means of proximating the skin prior to skin closure that are housed on or within the prior art of skin staplers.

Also, included are specific patents which are related to medical skin staplers.
1. U.S. Pat. No. 4,391,402
2. U.S. Pat. No. 4,406,392
3. U.S. Pat. No. 4,458,835
4. U.S. Pat. No. 4,591,086
5. U.S. Pat. Des. No. 269,459

### BRIEF SUMMARY OF THE INVENTION

The invention “Staple Align”, medical skin stapler with attached skin tissue forceps. The improvement to skin staplers will grasp skin bilaterally, simultaneously with equal tension for incisional and or injuries to disunited skin. Precise proximation prior to stapling for achieved skin closure, for increase healing and decreased scarring.

“Staple Align”, also allows for greater efficiency, by allowing a single handed operation for skin closure to be achieved. Most importantly the benefit to the patients well being. It is well known in the medical field that skin staplers are used routinely for skin closure.

It is a known practice there be an assist from another source for skin proximation to disunited skin prior to stapling by the use of one or two separate medical instruments, and or an assist from another individual.

### BRIEF DESCRIPTION OF THE SEVERAL VIEWS OF THE DRAWINGS

I, Claudette M. Ables, have invented a new design for a medical skin stapler as set for in the following specifications.

FIG. 1 Is a bottom view of a medical skin stapler showing my new design;

FIG. 2 Is a top view thereof;

FIG. 3 Is a rear view thereof;

FIG. 4 Is a front view thereof;

FIG. 5 Is a right side view thereof;

FIG. 6 Is a left side view

FIG. 7 Is a side view of medical skin stapler without forceps thereof;

FIG. 8 Is a bottom of medical skin staplers with staples without forceps thereof;

FIG. 9 Is a top of medical skin stapler without forceps.

FIG. 10 Is a working design view of improved stapler;
a. Is a top side view;
b. Is a body inside view;
c. Is a partial side view of the nose end of body showing new design;
d. Is a partial front view of nose end showing new design;

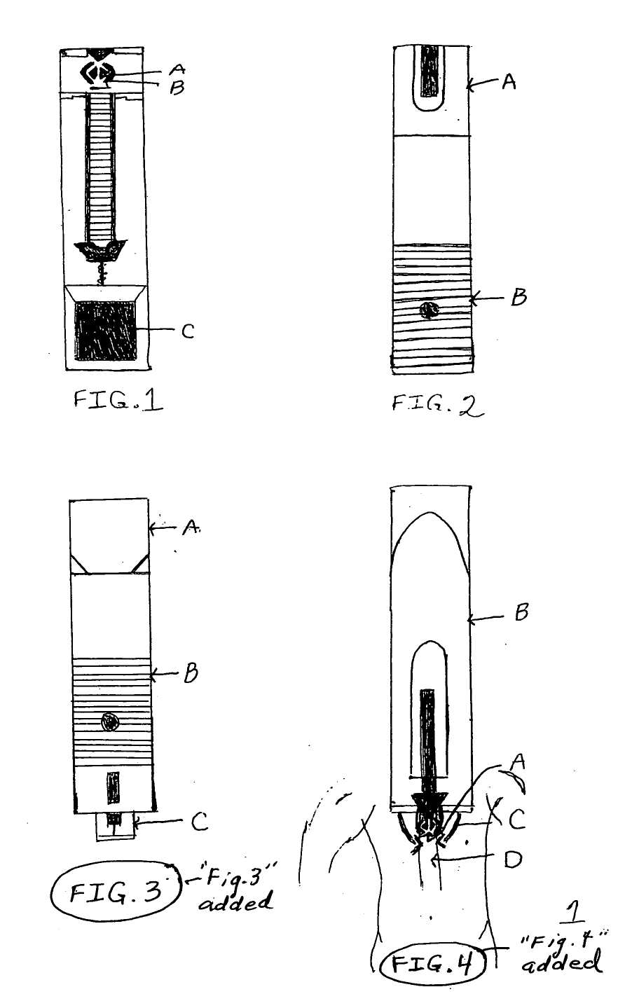

I claim: The ornamental design for a medical skin stapler with skin stapler with skin tissue forceps as shown.
FIG. 1 Bottom view skin stapler showing new design/improvement to existing skin stapler U.S. Pat. Nos. 4,391,402; 4,406,392; 4,458,835; 4,591,086 and DES. No. 269,459, which doesn't have a means for proximating disunited skin prior to stapling disunited skin. My improvements,
a. Single toothed
b. Double-toothed forceps for proximating disunited skin bilaterally
c. Trigger distal to nose end

FIG. 2 Top view skin stapler
a. Nose end
b. Body

FIG. 3 Back view skin stapler
a. Nose end
b. Body
c. Trigger

FIG. 4 Front view skin stapler
a. Nose end showing new design/improvement single and double toothed forceps bilaterally
b. Body
c. Staples
d. Skin

FIG. 5 Right side view skin stapler
a. Nose end showing forceps
b. Body
c. Trigger

FIG. 6 Left side view skin stapler
a. Nose end showing forceps
b. Staples
c. Disunited skin
d. Body
e. Trigger

FIG. 7 Left side view without forceps
a. Nose end
b. Body
c. Trigger
d. Staples

FIG. 8 Bottom side view
a. Nose end
b. Staples
c. Trigger

FIG. 9 Top side view of skin stapler
a. Nose end
b. Body

FIG. 10A Side view
10A-1 Top of skin stapler that fits over 10-B

FIG. 10B Body
10B-1 Nose end
10B-2 Trigger
10B-3 The downward motion when top is over body and trigger is grasped and squeezed for action of activation of forceps to grasp disunited skin and not to interrupt activation and implantation of sterile staple into skin. When complete and trigger released allows for release of forceps to skin and allows for repeat of process until closure achieved.

FIG. 10C Side end nose end
10C-1 Partial side view nose end showing forcep attached 10B

FIG. 10D Front view nose end
10D-1 Partial front view nose end showing forcep attached to 10B

1. What I claim as my invention is a medical skin stapler with attached skin tissue forceps
a) a medical skin stapler for grasping proximating disunited skin to incisional and or injuries to skin prior to stapling compromising;
b) a double toothed and single toothed forcep on each side of nose/bottom housed on and within skin stapler;
c) a grasping of hand held stapler and activating the trigger by grasping and squeezing the trigger in one action will activate the forceps bilaterally and simultaneously with equal tension will bring skin and forceps in union for precise proximation of disunited skin that is to be stapled;
d) a continued action of grasping squeezing manually hand held stapler will precock, release and impregnate sterile skin staple to skin;
e) a release of trigger will achieve stapling and release of forceps and skin bilaterally and simultaneously as to allow for repeat of process each and every time until complete skin closure is achieved.
